(19) United States
(12) Patent Application Publication
(10) Pub. No.: US 2001/0049097 A1
(43) Pub. Date: Dec. 6, 2001

(54) METHOD OF IMPROVED TRANSCRIPT EXTENSION OF NONCANONICAL TRANSCRIPTS USING MUTANT RNA POLYMERASES

(76) Inventor: RUI SOUSA, SAN ANTONIO, TX (US)

Correspondence Address: QUARLES & BRADY LLP, 411 E. WISCONSIN AVENUE, SUITE 2040, MILWAUKEE, WI, 53202-4497, US

(21) Appl. No.: 09/337,106

(22) Filed: Jun. 21, 1999

(51) Int. Cl. C12Q 1/68; C07H 21/02; C07H 21/04; C07H 21/00; C12P 19/34

(52) U.S. Cl. 435/6; 435/91.5; 536/23.1; 536/24.1; 536/25.3

(57) ABSTRACT

Disclosed is an improved method for transcript extension of nucleic acid molecules having at least one non-canonical nucleoside monophosphate, wherein an amount of a polyamine effective to enhance the extension rate is included in the reaction mixture. The polyamines spermidine and spermine have been found to be effective in the improved method when present in a concentration of at least 8.0 mM or 1.5 mM, respectively. The present invention includes a buffer for enhancing transcript extension of nucleotides having non-canonical substituents and an in vitro nucleotide synthesis reaction kit for enhanced extension of nucleotide transcripts having non-canonical substituents.

### CROSS-REFERENCE TO RELATED APPLICATIONS

Not applicable.

### STATEMENT REGARDING FEDERALLY SPONSORED RESEARCH OR DEVELOPMENT

This invention was made with United States government money in the form of NIH grant GM-52522-01. The United States government has certain rights in this invention.

### BACKGROUND OF THE INVENTION

There are a number of commonly used methods for in vitro synthesis of nucleic acid molecules, including both DNA and RNA. For example, one may use an in vitro transcription reaction to synthesize RNA from a DNA template present in the reaction. T7-type RNA polymerases, such as T7 RNA polymerase, T3 RNA polymerase, or SP6 RNA polymerase are commonly used in such reactions, although many other RNA polymerases may also be used. Synthesis of RNA is usually, but not always, de novo (i.e., the template is unprimed). Initiation of RNA transcription usually, but not always, occurs at a “promoter” or “promoter sequence,” a sequence in the template that is specifically recognized by the RNA polymerase.

A variety of procedures that employ in vitro nucleic acid synthesis to characterize nucleic acid molecules, including both RNA and DNA, are known to the art.

Characterization of nucleic acid molecules is used in a number of applications. For example, genes that are implicated in a wide range of human, animal, and plant disease can be identified and characterized. Within a single gene, numerous mutations are being identified that are correlated with particular pathological conditions. Characterization of nucleic acids is also useful in many other areas, including forensics, paternity testing, animal and plant breeding, tissue typing, and biological research. Although many methods for detecting both known and unknown mutations have been developed, progress in developing new, better, and faster methods for characterizing nucleic acids is critical to our ability to capitalize on information obtainable from nucleic acid sequences.

Although most methods for sequencing nucleic acids employ DNA polymerases, T7 RNA polymerase (RNAP) and SP6 RNAP have been used in transcription sequencing of DNA templates beginning at the T7 or SP6 promoter sequence, using 3′-deoxyribonucleoside-5′ triphosphates and Q-Beta replicase for sequencing single-stranded RNA templates. Also, 3′-O-methyl-ribonucleoside-5′-triphosphates have been used for sequencing DNA templates with E. coli RNA polymerase. However, none of these techniques is commonly used at present, in part because it is difficult to obtain the 3′-deoxy and 3′-O-methyl-ribonucleoside-5′-triphosphate substrates, and the readily available 2′-3′ dideoxyribonucleoside-5′-triphosphates are not substrates for wild-type RNA polymerases.

Nucleic acid polymerases can be classified according to their template specificities (RNA or DNA), substrate specificities (rNTPs or dNTPs), and mode of initiation (de novo or primed). These classifications are generally based on the specificity or activity the enzyme displays in vivo in fulfilling its biological function.

Polymerases can display novel activities in vitro, although such activities take place with reduced efficiency or under non-physiologic conditions. For example, E. coli DNA-directed DNA polymerase I can use RNA as a template but the polymerase's Kₘ for dNTP is about 100 times higher when RNA is used as a template than when DNA is used as the template. T7 DNA-directed RNA polymerase can also use RNA as a template. Relaxed in vitro template specificity is fairly common among polymerases.

In contrast, polymerase substrate specificity is generally extremely stringent. T7 DNAP, for example, displays at least 2000-fold selectivity for dNTPs over rNTPs, even in the presence of Mn⁺⁺, which relaxes the ability of the polymerase to discriminate between dNTPs and ddNTPs.

U.S. Pat. No. 5,849,546 discloses T7-type RNAPs bearing a mutation that reduces the ability of the polymerases to discriminate between canonical and noncanonical nucleotides, and methods for synthesizing a nucleic acid molecule having at least one non-canonical nucleotide using a T7-type RNAP that bears a mutation that confers reduced discrimination between substrates.

Although the method disclosed in U.S. Pat. No. 5,849,546 facilitates incorporation of non-canonical substrates into nucleic acids, the rate at which transcripts containing a high percentage of non-canonical NMPs are extended is dramatically reduced, presumably due to the effects of the 2′ substituents on the conformation of the hybrid formed between the transcript and the template.

What is needed in the art is a method for synthesizing nucleic acids in vitro using non-canonical substrates that gives increased rates of transcript extension.

### BRIEF SUMMARY OF THE INVENTION

One aspect of the present invention is a method of increasing the rate of transcript extension of a nucleic acid molecule having at least one non-canonical nucleotide comprising the steps of:

a) incubating a template nucleic acid in a reaction mixture under nucleic acid synthesis conditions, the reaction mixture containing (i) a mutant RNA polymerase, wherein the polymerase is selected from the group consisting of an SP6 RNA polymerase comprising an altered amino acid at position 631 and T7 RNA polymerase comprising an altered amino acid at position 639, wherein said polymerase has reduced discrimination between canonical and non-canonical nucleoside triphosphates, (ii) at least one non-canonical nucleoside triphosphate, wherein said non-canonical nucleoside triphosphate is incorporated into the synthesized nucleic acid in place of only one canonical nucleoside triphosphate, and (iii) a polyamine present at a concentration effective to enhance the rate of transcript extension, the polyamine selected from the group consisting of spermidine at a concentration of at least about 8 mM and spermine at a concentration of at least about 1.5 mM; and b) obtaining the synthesis of a nucleic acid molecule having at least one non-canonical nucleotide.

Another aspect of the present invention is a buffer for enhancing transcript extension comprising at least one non-canonical nucleotide, the buffer comprising either spermidine or spermine, wherein the spermidine or spermine is present at a concentration sufficient to give a final concentration of at least about 8 mM spermidine or at least about 1.5 mM spermine when the buffer is diluted to give appropriate nucleic acid synthesis conditions.

In another embodiment, the present invention is a kit for in vitro synthesis of a nucleic acid molecule having at least one non-canonical nucleoside triphosphate comprising a mutant nucleic acid polymerase having reduced discrimination between canonical and non-canonical nucleoside triphosphates, a protocol describing conditions under which the synthesis can be conducted, and a polyamine selected from the group consisting of spermidine and spermine present in an amount effective to give at least about 8 mM spermidine or at least about 1.5 mM spermine when the buffer is used according to the protocol.

### BRIEF DESCRIPTION OF THE SEVERAL VIEWS OF THE DRAWINGS

FIG. 1 Panels A and B are autoradiograms of polyacrylamide gels showing radiolabeled, electrophoretically separated RNA transcripts.

FIG. 2. Panels A and B are autoradiograms of polyacrylamide gels showing radiolabeled, electrophoretically separated RNA transcripts; panel C is a schematic representation of various types of promoter structure.

### DETAILED DESCRIPTION OF THE INVENTION

The single-subunit RNA polymerases encoded by the T7 and SP6 bacteriophage are widely used to prepare RNAs by in vitro transcription for a variety of applications (1). A T7 RNA polymerase mutant that incorporates NMPs bearing non-canonical groups at the ribose 2′-position into RNA was recently identified (2). This mutant facilitates preparation of 2′-modified RNAs for structural and structure-function studies (3-6), as well as for other applications (7).

The terms mutant polymerase, template, nucleotide, nucleoside, incorporation, canonical, non-canonical, and T7-type RNA polymerases, and any term not expressly defined herein, have the meanings provided by the definitions in U.S. Pat. No. 5,849,546, which is incorporated by reference herein in its entirety.

By “mutant polymerase” is meant a nucleic acid polymerase that has at least one altered amino acid compared to the corresponding wild-type polymerase, wherein said mutation or alteration results in the mutant polymerase having reduced discrimination between non-canonical and canonical nucleoside triphosphates as substrates.

As used herein, a “mutant polymerase having reduced discrimination between non-canonical and canonical nucleoside triphosphates as substrates” is a polymerase for which the ability to discriminate between canonical and non-canonical nucleosides is reduced by at least about 10-fold relative to the corresponding wild-type enzyme, wherein the respective values for discrimination between canonical and non-canonical substrates is calculated using the average of the k_{cat}/Kₘ values for all four rNTPs and all four dNTPs.

By “template” we mean a macromolecular pattern or mold for the synthesis of another macromolecule, composed of a sequence of nucleotides, either rNTPs or dNTPs, that serves to specify the nucleotide sequence of another structure.

“Nucleotide” refers to a base-sugar-phosphate compound. Nucleotides are the monomeric subunits of both types of nucleic acid polymers, RNA and DNA. “Nucleotide” refers to ribonucleoside triphosphates, rATP, rGTP, rUTP and rCTP, and deoxyribonucleoside triphosphates, such as DATP, dGTP, dTTP, dCTP.

As used herein, “nucleoside” refers to a base-sugar combination without a phosphate group. “Base” refers to the nitrogen-containing base, for example adenine (A), cytidine (C), guanine (G) and thymine (T) and uracil (U).

“Incorporation” refers to becoming a part of a nucleic acid polymer. There is a known flexibility in the terminology about incorporation of nucleic acid precursors. For example, the nucleotide dGTP is a deoxyribonucleoside triphosphate. Upon incorporation into DNA, it becomes a dGMP, or deoxyguanosine monophosphate moiety. Although there is no dGTP molecule in DNA, one may say that one incorporates dGTP into DNA.

As defined herein, a “canonical” nucleoside triphosphate for an RNA polymerase (“RNAP”) consists of any ribonucleoside-5′-triphosphate (“rNTP” or “NTP”) which has an hydroxyl group at the 2′-position of the sugar, including, but not limited to, the four common ribose-containing substrates for an RNA polymerase -ATP, CTP, GTP and UTP. A 2′-deoxyribonucleoside-5′-triphosphate (“dNTP”) which has hydrogen at the 2′-position of the sugar, including, but not limited to, the four common deoxyribose-containing substrates (DATP, dCTP, dGTP and dTTP, also known as “TTP”) for a DNA polymerase (“DNAP”) is defined herein as a “non-canonical” nucleoside-5′-triphosphate or a “non-canonical NTP” or a “non-canonical nucleotide” or a “non-canonical deoxynucleotide” or a “non-canonical triphosphate” or a “non-canonical substrate” for an RNA polymerase. On the other hand, a “canonical” nucleoside triphosphate for a DNAP consists of any dNTP which has a hydrogen at the 2′-position of the sugar, while an rNTP is defined as a “non-canonical NTP” or a “non-canonical nucleotide” or a “non-canonical substrate” for a DNAP. The terms “canonical” and “non-canonical” are meant to be used herein only with reference to the 2′ position of the sugar. Thus, as defined herein, 2′,3′-dideoxynucleoside-5′ triphosphates (“2′,3′-ddNTPs” or “ddNTPs”) are “noncanonical” substrates for an RNAP, but are defined as “canonical” for a DNAP. Further, any other substituent than an hydroxyl group at the 2′-position of ribose or a hydrogen at the 2′-position of deoxyribose, including, but not limited to, a fluorine (“F” or “fluoro” group) or an amino group, would be defined as “non-canonical” for both RNAPs and DNAPs herein. The terms “canonical” or “non-canonical” also are not meant to refer to the nucleic acid bases attached to the sugar moieties. Thus, for example, other natural or modified nucleic acid bases attached to the 1′-position of ribose-5′-triphosphate would still be defined as “canonical” herein.

By “T7-type RNA polymerases” we mean T7, T3, φI, φIIH, W31, ghl, Y, A1122, SP6 and mitochondrial RNAPs.

In most cases, the mutant polymerase can be used just as one would use the corresponding wild type enzyme in standard transcription buffers. However, the standard reaction conditions commonly used with native or wild type polymerases having high substrate discrimination do not allow mutant polymerase-catalyzed extension of transcripts having non-canonical substituents to proceed at an efficient rate. Provided that rGTP is not replaced, activity in a standard transcription buffer with a supercoiled template containing a consensus promoter is reduced ˜2-fold when a single rNTP is replaced by a dNTP, a 2′-NH₂-NTP, or a 2′-F-NTP (2,14). Even more drastic activity reductions are obtained when 2, 3, or 4 of the rNTPs in the transcription reaction are replaced by NTPs with non-canonical 2′-groups (2,14,20).

Characterization has shown that reduced extension of transcripts having non-canonical 2′-groups is due, at least in part, to a conformational effect on the transcript:template hybrid. Efficient transcript extension appears to require that the transcript:template hybrid in the active site assume an A-type conformation (20). Since the mutation eliminates discrimination of the chemical character of the 2′ substituent, but does not eliminate the requirement for a particular conformation of the transcript:template hybrid, transcripts which are heavily modified with non-canonical 2′-groups become poor substrates for transcript extension if the modifications favor assumption of a distinct (B-type) conformation in the transcript:template hybrid.

It may also be that conformational effects interfere with the incorporation step. While the mutation may eliminate direct discrimination of the chemical character of the 2′-substituent, it does not eliminate discrimination of the effects of this group on the ribose conformation of the NTP. Ribose conformation is a function of the electronegativity of the 2′ substituent, with more electronegative substituents preferring the C3′-endo conformer seen in A-form nucleic acid structures, while less electronegative substituents prefer the C2′-endo conformer seen in B-type helices (21,22). In experiments with a set of NTPs differing in their preferred ribose pucker and the H-bonding character of their 2′-substituents, the wild type enzyme strongly preferred NTPs whose substituents could act as H-bond donors or acceptors, while the mutant enzyme displayed a weak, residual preference which followed the C3′-endo content of the NTP ribose (14).

It was hypothesized that these putative conformational obstacles to synthesis of nucleic acids heavily modified with non-canonical 2′-substituents might be reduced by employing in the reaction mixture reagents that stabilize A-form helical structures. Such compounds include, for example, methanol, ethanol, trifluoroacetic acid, cobalt hexamine, spermine and spermidine.

The effect of various compounds was tested, as described below in the examples, by making certain modifications to standard reaction conditions typically used for transcription reactions and evaluating the effect of the modifications on the rate of transcript extension with a mutant polymerase in reaction mixtures having non-canonical nucleotides.

By “standard reaction conditions” it is meant reaction conditions suitable for transcript extension using canonical nucleotides. In general, in vitro transcription reactions are generally buffered with 40 mM Tris-HCl or Tris-acetate buffer pH 8.0. Magnesium is supplied as MgCl₂ or Mg-acetate at a concentration of from 5-15 mM. To maintain enzyme stability, 5 mM DTT is included. Optionally, EDTA is added at a concentration of 1 mM. Additional components may be added as well, including, for example, pyrophosphatase or Tween 20.

Surprisingly, inclusion of spermidine or spermine in reaction mixtures having two or more rNTPs substituted with non-canonical nucleotides stimulated the activity of a mutant polymerase (Y639RNAP) at concentrations that are inhibitory in reaction mixtures containing all four rNTPs (i.e., lacking non-canonical nucleotides). Inclusion of spermidine or spermine at high concentrations allowed transcript extension of longer nucleic acids molecules in which numerous 2′-hydroxyls had been replaced by non-canonical substituents.

In the examples below, the addition of spermidine or spermine at concentrations of at least about 8 mM or at least about 1.5 mM, respectively, were found to increase the rate of Y639RNAP-catalyzed transcript extension in reaction mixtures in which more than one rNTP had been replaced by a non-canonical nucleoside triphosphate.

As the examples below demonstrate, the concentration of spermidine or spermine effective to enhance transcript extension of non-canonical transcripts may vary as a function of the template used. When a plasmid such as pT75 is used as the template, spermine concentrations of 4 mM or greater inhibit transcription with all rNTP and dNTP combinations, and spermidine concentrations of at least about 8 mM allow increased transcription. When a relatively short polynucleotide is used as the template, reactions containing 3 dNTPs and 1.6 mM spermine gave two-fold greater transcription than reaction mixtures with 8 mM spermidine, and concentrations of up to 13 mM spermine were stimulatory. It is therefore envisioned that spermine concentrations of 1.5 mM, 5 mM, or even as much as 15 mM could be used in the practice of the present invention. Similarly, spermidine concentrations of 8 mM, 15 mM, or even as much as 20 mM could be successfully employed in enhancing mutant polymerase activity, thereby increasing the rate of transcript extension.

It is reasonably expected that other polyamines could be substituted for spermidine or spermine to enhance extension of transcripts having non-canonical substituents. By a polyamine, it is meant a compound that contains two or more positively charged amino groups. Other polyamines that may be suitable in the practice of the present invention include any naturally occurring polyamines or synthetic polyamines including, but not limited to, the synthetic polyamines as disclosed in Frugier et al. (Nucl. Acids Res. 22:2784-2790, 1994). Using the teachings of the present invention, the concentration of a polyamine needed to give optimal transcript extension using non-canonical nucleotides could be determined for any suitable polyamine.

In addition to a suitable polyamine, reagents that enhance T7 RNAP polymerase activity in general, or which enhance the extension of non-canonical transcripts in particular, may also be included in the transcription reaction. Varying concentrations of Mn⁺⁺, which may favor utilization of substrates or transcripts of non-canonical structure by a distinct mechanism (20), was tested to determine the effect on the mutant polymerase activity, as described below. Reagents such as non-ionic detergents, acetate, and pyrophosphatase, which are not expected to specifically enhance the use of non-canonical substrates, but which have been reported to generally enhance T7 RNAP activity (23), were also evaluated.

The results of experiments described below led to the development of a buffer that affords surprisingly increased levels of the mutant Y639F T7 RNAP polymerase activity. An example of suitable reaction conditions for the practice of the present invention that are not inhibitory in reactions with canonical substrates and which greatly promote transcript extension with Y639 F and non-canonical substrates includes 40 mM Tris-acetate pH 8.0, 5 mM DTT, 1 mM EDTA, 10 mM Mg-acetate, 0.5 mM MnCl₂, 8 mM spermidine, and 1 U/μl pyrophosphatase. One skilled in the art will appreciate that one may vary the reaction conditions from those exemplified herein and still obtain an increased extension rate of transcripts having non-canonical nucleotides.

Inclusion of pyrophosphatase, a relatively expensive component, is not necessary unless high concentrations of RNA are being prepared. However, when high concentrations of RNA are being made, pyrophosphate accumulation may inhibit polymerase activity (25). When synthesizing RNA in high concentrations (i.e., transcript yields of greater than 1 mg/ml), one may include pyrophosphatase in reaction mixtures.

The method of the present invention increases rates of transcript extension relative to those obtained using transcription reaction mixtures that lack high concentrations of spermidine or spermine. The extent to which the rates are increased by the method of the present invention depends on the type of non-canonical substituents used in the reaction mixture, as well as the size and structure of the template.

Using the method of the present invention, transcript extension rates can be increased at least 2-fold, relative to rates obtained under similar conditions in a reaction mixture lacking spermidine or spermine. Preferably, the rate is increased by 5-fold or 10-fold, relative to rates obtained under similar conditions in a reaction mixture lacking spermidine or spermine. More preferably, the rate is increased by 20-fold or even as much as 40-fold or more, relative to rates obtained under similar conditions in a reaction mixture lacking spermidine or spermine.

It should be appreciated that the present invention can be practiced not only with Y639F RNAP, but with any T7-type or SP6 RNA polymerase bearing an analogous mutation that affects discrimination between substrates, as disclosed, for example, in U.S. Pat. No. 5,849,546.

The following nonlimiting examples are intended to be purely illustrative.

### EXAMPLES

### Expression and Purification of Mutant T7 Polymerase

An expression vector containing the mutant T7 polymerase (Y639F) (16) was prepared by ligating a gene encoding the polymerase to linearized plasmid vector pDPT7. This vector was constructed by subcloning the BamHI fragment from pAR 1219, which was originally constructed in F. W. Studier's laboratory (18), into the pUC119 phagemid. Higher levels of expression of T7 RNAP is obtained with pDPT7 than with pAR 1219. Preferably, the gene is expressed in a protease deficient strain such as BL21, to allow recovery of the enzyme in intact form. To ensure the stability of the plasmid, cell culture and polymerase expression is conducted as follows.

Approximately 12-18 hours before inoculating a culture, cells are streaked out on LB agar plates containing 100 mg/ml ampicillin to obtain a plate with ˜100-10,000 colonies. The plates should not be stored after growth. Immediately before inoculation, the cells from a freshly grown plate are harvested and suspended in 5-10 mls. of media, which is used to inoculate 1-4 liters of LB containing 100 μAg/ml ampicillin. After the culture has reached an O.D. 600 of 0.3-0.6, IPTG is added to a final concentration of 125 μg/ml to induce expression.

Four to six hours after ITPG was added, the cells were harvested by centrifugation. The cell pellet was resuspended in chilled lysis buffer (25 mM EDTA, 20 mM Tris-Cl pH 8.0, 10% sucrose, 10 mg/ml lysozyme) and incubated at 40° C. for 30 min. Cells were subjected to freeze-thaw cycling to break the cells. Cell debris was removed by centrifugation at 40° C. (15 min. top speed in minifuge, or 15-20K for 30 min. in a Sorvall SS-34 rotor for larger scale preparations). The supernatant was applied to a Whatman P-11 phosphocellulose column (˜2-3 mls. of packed resin for every 10 mls. of supernatant) equilibrated with 25 mM EDTA, 10% sucrose, 20 mM Tris-Cl pH 8.0; washed with several column volumes of same the buffer +0.15 M NaCl. Proteins were eluted with either a step of buffer +0.4 M NaCl or a gradient of buffer +0.15 M NaCl to buffer +0.5 M NaCl. T7 RNAP elutes between 0.3 and 0.4 M NaCl. By this method, yields of up to 30 mg protein/liter culture were obtained. The enzyme is suitable for most purposes after this single chromatographic step. If greater purity is desired, the protein can be further purified by anion exchange chromatography on any strong anion exchange resin (i.e. DEAE, Pharmacia Mono Q,), and/or by sizing chromatography on gel exclusion media of appropriate MW cutoff (Pharmacia Sephacryl S-200) as described (19)

### ENZYME STORAGE

For storage at −20° C., the eluate from the phosphocellulose column may be dialyzed into a storage buffer (0.5 M NaCl, 20 mM sodium phosphate pH 8.0, 5 mM DTT, 1 mM EDTA, 50% glycerol). The mutant polymerase generally stores well, but may begin to lose activity after several months. If loss of activity is due to oxidation of the reducing agent in the storage buffer, activity may be increased by a fresh aliquot of DTT.

### Activity of Mutant RNAP in Various Transcription Buffers

In general, transcription reaction mixtures comprised about 40 mM Tris-HCl or 40 mM Tris-Acetate pH 8.0, 5 mM DTT, 5-15 mM MgCl₂, 0.1 mCi/ml of alpha-³²P GTP, and various combinations of rNTPs and dNTPs at initial concentrations of 0.5 mM and 1.0 mM, respectively. The reaction mixtures included supercoiled or PvuI-linearized pT75 DNA (10⁻⁸ M) as the template and mutant T7 Y639F polymerase (10⁻⁷ M). Reactions were typically conducted in 50 microliter reaction volumes and incubated at 37° C. Aliquots were removed after 5, 10, and 20 minutes of incubation and the reactions were quenched by adding a solution containing 95% formamide, 20 mM EDTA, 0.01% xylene cyanol. The reaction products were resolved by electrophoresis on denaturing gels containing 6% acrylamide, 0.6% bis-acrylamide, 6 M urea, 1×TBE. The gel was analyzed using a Molecular Dynamics Phosphorimager to determine the rates of incorporation of radioactive GTP into specific transcripts.

To evaluate the effect of A-form stabilizing compounds on polyamines on transcript extension by a mutant T7 RNAP, the activity of the RNAP was evaluated using standard transcription conditions (15 mM MgCl₂, 40 mM Tris-HCl pH 8.0, 1 mM EDTA, and 5 mM DTT), or standard transcription reaction conditions plus varying concentrations of ethanol, methanol, trifuoro acteic acid, cobalt hexamine, spermine, or spermidine.

In addition to evaluating A-form stabilizing compounds, we examined the effects of varying the concentration of Mg²⁺ and Mn²⁺, substituting acetate for chloride as the main reaction counteranion, adding 0.1% tween-20, and adding pyrophosphatase.

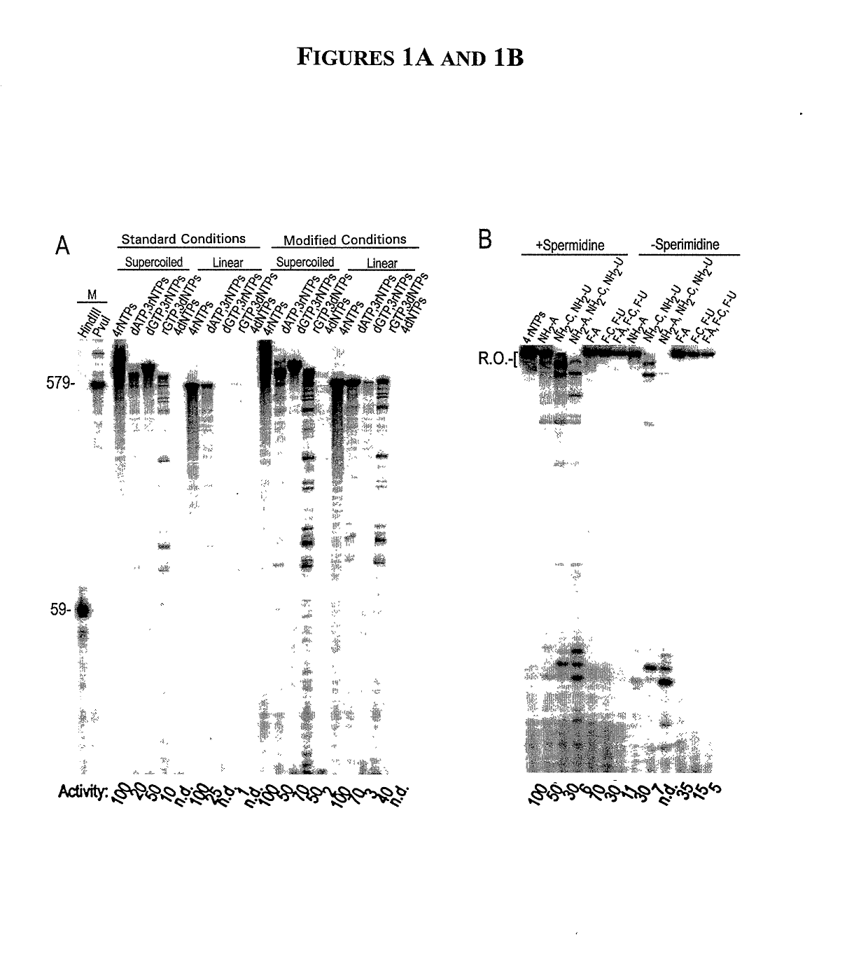

With reference to FIG. 1A, reactions in lanes labeled ‘M’ were carried out with wild type polymerase, 4 rNTPs, and either Pvu I- or Hind III-restricted pT75 to generate RNA markers ˜579 or ˜59 bases in length, respectively. Values below each gel lane give the relative rates of synthesis of runoff or long (>˜600 base) transcripts on linearized or supercoiled templates, respectively, normalized to a value of 100 for the 4 rNTP reactions. FIG. 1A compares the relative activities of Y639F in typical polymerase reaction conditions (15 mM MgCl₂, 40 mM Tris-HCl pH 8.0, 1 mM EDTA, and 5 mM DTT) and in a modified reaction mixture (40 mM Tris-acetate pH 8.0, 5 mM DTT, 1 mM EDTA, 10 mM Mg-acetate, 0.5 mM MnCl₂, 8 mM spermidine, and 1 U/μl pyrophosphatase) in reactions having various combinations of rNTPs and dNTPs with supercoiled or linearized templates.

With reference to FIG. 1B, transcripts were synthesized using Hind III-cut pT75 as the template with 2′-NH₂- or 2′-F NTPs as indicated replacing the corresponding rNTPs in the reaction. Reactions were conducted with 8 mM or 0 mM spermidine, as indicated. FIG. 1B compares the activity of Y639F in the presence or absence of spermidine, in reactions with 4 rNTPs or different combinations of rNTPs and 2′-F- or 2′-NH₂-NTPs.

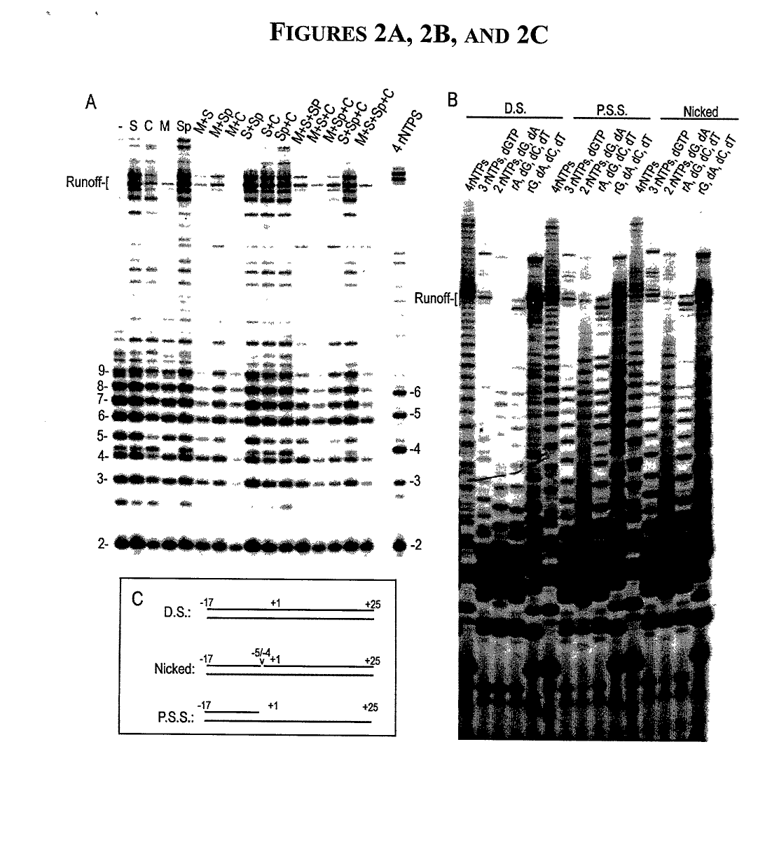

A 42 base-pair synthetic template carrying a consensus T7 promoter was tested for its effect on Y639F T7RNAP activity by measuring the synthesis of 25 base runoff transcripts in reaction mixtures containing 10 mM MgCl₂, 40 mM Tris pH 8.0, 1 mM EDTA, 5 mM DTT, 0.5 mM MnCl₂ and either 3 dNTP+rGTP or 4 rNTP reactions (FIG. 2A). In addition, the reactions contained the following additives as indicated in individual lanes: S=8 mM spermidine, C=1 mM cobalt hexammine chloride, M=20% methanol, Sp=1.6 mM spermine. The polymerase and T7 promoter were included at concentrations of about 10⁻⁷ M. Reaction products were subjected to electrophoresis on denaturing gels containing 20% acrylamide, 2% bis-acrylamide, 6 M urea, 1×TBE. Runoff transcripts (R.O.) are heterogeneous in length because T7 RNAP can terminate extension ˜1-3 bases before reaching the end of the template or add 1 or more bases to the end of the runoff transcript. With reference to FIG. 2B, the effect of template structure on synthesis of runoff transcripts in a reaction mixture comprising 40 mM Tris-acetate pH 8.0, 5 mM DTT, 1 mM EDTA, 10 mM Mg-acetate, 0.5 mM MnCl₂, 8 mM spermidine, and 1 U/μl pyrophosphatase and the indicated combinations of rNTPs and dNTPs were evaluated using promoters with template sequences identical to that used in FIG. 2A but which are either fully double-stranded, partially single-stranded, or nicked (FIG. 2C).

### Addition of High Levels of Polyamines Enhances Activity

In reactions containing 4 rNTPs and no non-canonical nucleotides, nucleotide incorporation was inhibited by ethanol, methanol, trifluoroacetic acid, and cobalt. Spermine and spermidine had minimal effects at low concentrations and were inhibitory at high concentrations. The existence of a concentration optimum in reactions with 2 or more dNTPs appeared to be the net result of two countervailing tendencies: the compounds are generally inhibitory of the transcription reaction, but also limit the degree to which replacement of rNTPs with dNTPs reduces incorporation rates. Of all tested compounds, the polyamines, particularly spermidine and spermine, were the most effective at stimulating incorporation in reactions with multiple dNTPs. No combination of the tested compounds was superior to polyamines alone.

Addition of tested polyamines had by far the most stimulatory effect on the activity of the mutant enzyme in reactions in which 3 rNTPs were replaced by dNTPs. The concentration optima for the polyamines in a reaction appears to be a function of two opposed effects. Spermine or spermidine concentrations in excess of ˜1 or 10 mM, respectively, were generally inhibitory of activity as assessed in a reaction with 4 rNTPs and plasmid templates. However, these reagents decreased the degree to which activity was reduced in reactions with non-canonical substrates. The net effect led to up to approximately a 5-fold increase in activity in reactions with 3 rNTPs and plasmid templates, with concentration optima around 8 mM for spermidine and 1-2 mM for spermine.

These experiments led to the discovery of reaction conditions that surprisingly do not inhibit reactions with canonical substrates and that afford greatly enhanced transcript synthesis with Y639F and non-canonical substrates. An example of such conditions includes a reaction mixture comprising 40 mM Tris-acetate pH 8.0, 5 mM DTT, 1 mM EDTA, 10 mM Mg-acetate, 0.5 mM MnCl₂, 8 mM spermidine, and 1U/μl pyrophosphatase.

The stimulatory effects of the polyamines may be largely attributable to stabilization of an A-conformation in transcript:template hybrids carrying transcripts heavily substituted with non-canonical NMPs, the riboses of which favor a C₂′-endo conformation. It is also possible that the polyamines may enhance formation of the catalytically correct geometry in the template:transcripte.NTP complex, especially if the NTP must stack on the transcript and base-pair with the template so as to extend the helix conformation of the transcript:template hybrid.

The stimulatory effect of the polyamines may therefore differ, depending on the template structure and the nucleoside triphosphate used.

### Substrate and Template Effect on Polymerase Activity

The effect of substrates bearing larger substituents (2′-O-Methyl groups) on Y639F activity was tested using a modified buffer (40 mM Tris-acetate pH 8.0, 5 mM DTT, 1 mM EDTA, 10 mM Mg-acetate, 0.5 mM MnCl₂, 8 mM spermidine, and 1 U/μl pyrophosphatase). Incorporation of these substrates was relatively inefficient and synthesis of 59 base runoff transcripts from Hind-III linearized pT75 in reactions with a single 2′-O-Me-NTP averaged ˜20-fold lower than in reactions with 4 rNTPs (data not shown). However, replacement of rGTP with 2′-O-Me-GTP eliminated synthesis of runoff transcripts altogether.

Transcription reactions employing plasmid pT75 DNA as the template were inhibited by spermine in concentrations of ≧4 mM in all combinations of rNTPs and dNTPs, and spermidine at a concentration of 8 mM was most effective. In contrast, in reactions employing a 42 bp template identical in sequence to pT75 from −17 to +25, spermine concentrations of up to 13 mM were shown to be stimulatory in reactions with 3 dNTPs; at 1.6 mM, spermine gave 2-fold greater runoff synthesis than 8 mM spermidine (FIG. 2A, and data not shown).

Replacement of rGTP with dGTP reduces activity on a linear template to a greater extent than observed with a supercoiled template (FIG. 1A). A template-structure dependent effect of similar magnitude is not observed when rATP is replaced with DATP, nor when 3 rNTPs are replaced with dNTPs but rGTP is retained. This is probably due to the G-richness of the initially transcribed sequence (ITS; the +1 to +6 sequence is GGGAGA) so that replacement of rGTP with a non-canonical substrate is especially disruptive of the poorly processive initial transcription reaction. Supercoiling, by facilitating promoter opening and stabilizing the initial transcription complex (29), may mitigate an inefficient initial transcription reaction. Other template structures (nicked or partially single-stranded promoters) which facilitate promoter opening and stabilize the initial transcription complex also enhance synthesis of runoff transcripts in reactions in which rGTP is replaced with dGTP (FIG. 2B) The improvements in synthesis of modified nucleic acids obtained by using a modified buffer (40 mM Tris-acetate pH 8.0, 5 mM DTT, 1 mM EDTA, 10 mM Mg-acetate, 0.5 mM MnCl₂, 8 mM spermidine, and 1 U/μl pyrophosphatase), relative to a standard buffer, are large. For example, the synthesis of a 579 base transcript in a reaction with 3 dNTPs was increased ˜40-fold when the modified buffer was used (FIG. 1A). In the spermidine-containing buffer it was also possible to synthesize long transcripts from supercoiled templates in reactions with 4 dNTPs, and to synthesize runoff transcripts in reactions with dGTP or 3 NH₂-NTPs, whereas when the spermidine or spermine was omitted from the reaction, no detectable runoff transcript synthesis is obtained with such NTP mixes (FIG. 1, 2; ref. 2). The referred to differences in synthesis are differences in rates, rather than yields, which means that even when rates are appreciably reduced (i.e. ˜20-fold reductions with 2′-O-Me-NTPs), it should usually be possible to obtain good yields by carrying out reactions for longer times and/or at higher template/polymerase concentrations. These reaction conditions should therefore considerably broaden the utility of the mutant enzyme for synthesizing modified nucleic acids. For the convenience of being able to use a single reaction buffer for most reaction conditions, a versatile buffer was defined so as to give high activity in reactions with a variety of substrates and templates. In specific cases it may be useful to modify these conditions.

When designing a suitable transcript extension buffer, the following factors about how buffer components, template structure, substrate structure, and concentrations affect synthesis must be kept in mind:

1. The degree to which A-type geometry is disfavored in the transcript:template hybrid is a function of base structure, the intrinsically preferred ribose pucker of the nucleotides in the 3′ region of the transcript, and the number of non-canonical riboses in that part of the transcript (20). For example, incorporation of dAMP into the transcript is expected to destabilize A-type geometry to a greater degree than incorporation of dGMP (20, 21). Of the different 2′-substituents examined the preferred ribose pucker of the 2′-F NTPs most strongly favors the A-type conformation. The preferred pucker of the 2′-NH₂-NTPs most strongly disfavors A-type conformation and favors B-type conformations, while 2′-H-NMPs also favor B-type over A-type, but not as strongly as the NH₂-NMPs do (21, 22).

The reduction in transcript extension efficiency is consistent with the predicted effects of these nucleotides on helix conformation and the proposal that disruption of A-type helix geometry limits transcript extension. Thus replacement of rGTP with dGTP may limit initiation, but replacement of rATP with DATP limits transcript extension more as evidenced by the presence of increased premature termination in the reactions with DATP (FIG. 1A). Replacing 3 rNTPs with dNTPs decreases the efficiency of transcript extension even more as revealed by even more premature termination (transcripts shorter than the runoff; FIG. 1).

The ˜40-fold increase in synthesis of runoff transcripts in 3 dNTP reactions in the modified buffer of FIG. 1A is largely due to increased efficiency of transcript extension as evidenced by a reduced proportion of premature termination. Premature termination is greatest in the reactions with the 2′-NH₂ NTPs, and is least in the reactions with 2′-F NTPs (FIG. 1B). Because spermidine is supposed to act primarily by stabilizing A-type helix geometry in a hybrid whose nucleotide structure intrinsically disfavors such geometry, spermidine addition should have the greatest effects when this geometry is most disfavored. This is what is observed: the modified buffer is more stimulatory in reactions with 3 or 4 dNTPs than in reactions with 1 DNTP (FIG. 1A). Spermidine also has larger effects in reactions with 2′-NH₂ NTPs than in reactions with 2′-F NTPs (FIG. 1B).

Because the effect of spermidine is a function of the ribose and base structure of the non-canonical substrates being used, as well as of their fractional representation in the transcript, higher concentrations of spermidine (or spermine, see below) should be screened when synthesis with particular non-canonical substrates is too low, especially if the preferred ribose pucker of those substrates strongly disfavors A-type helix geometry and if a heavy degree of substitution in the transcript is sought. Because a reduction in transcript extension efficiency amounts to an effective reduction in processivity, the consequences for transcript yields increase as transcript lengths increase. This also means that the stimulatory effects of the polyamine-containing reaction mixture increase as transcript length increases. For example, synthesis of 59 base transcripts from Hind-III cut pT75 in a reaction with 3 dNTPs+rGTP is stimulated ˜8-fold by use of the polyamine-containing reaction mixture (not shown). However, synthesis of a 579 base runoff transcript from pT75 in a reaction with 3 dNTPs is stimulated ˜40-fold in the polyamine-containing reaction mixture (FIG. 1A).

2. While ˜8 mM spermidine is the most generally useful additive in reactions with plasmids, spermine is a more potent stimulator in reactions with short, synthetic templates (FIG. 2A). This probably reflects the fact that polyamines condense and aggregate nucleic acids (this may also contribute to the stimulatory effects of these polyamines by stabilizing short transcript:template hybrids during initiation). Since aggregation is favored by increasing molecular size and concentration, it is less of a problem with smaller or more dilute templates. When using small synthetic promoters or purified restriction fragments as templates, the use of spermine (1.5-3 mM) or higher concentrations of spermidine (≧8 mM) should be tested since this may provide greater stimulation of activity.

3. The initial transcription reaction (from +1 to ˜+8) is poorly processive and extremely sensitive to anything that perturbs catalysis, whether due to incorporation of non-canonical substrates or a non-consensus ITS (1,2). The barrier to incorporation of non-canonical substrates during initial transcription can, however, be mitigated by using templates which facilitate promoter opening and stabilize the initial transcription complex (FIG. 1, Ref. 3), so such templates may be useful when using non-consensus ITSs or when incorporating non-canonical substrates during initial transcription. However, reannealing of non-template and template strands is important for later stages of transcription such as promoter release and RNA displacement (28). On nicked or partially single stranded templates both promoter release and the processivity of the elongation complex are less efficient. If non-canonical substrates are not incorporated until late in initiation (after ˜+6) or if long transcripts are sought, the use of fully double-stranded templates is recommended. The initial transcription reaction is also characterized by high apparent NTP Kₘ values, especially with non-consensus ITSs (31). Increases in the concentrations of specific NTPs (up to a few mM) should also be screened if synthesis with particular ITSs or substrates is inefficient.

4. Though transcription by T7 RNAP is less efficient with Mn⁺⁺ than Mg⁺⁺, the Mn⁺⁺ catalyzed reaction is less stringent in its substrate/transcript specificity (2,20). The mechanism of this reduced specificity is distinct from the effects of polyamines or the Y639F mutation (20). The presence of Mn⁺⁺ also increases the uniformity of incorporation of modified substrates during polymerization, as reported for example, by Tabor & Richardson (26) for enhancing the uniformity of ddNTP incorporation by T7 DNAP. Mn⁺⁺ concentration should be screened as a variable with substrates that are poorly incorporated or in efforts to improve the uniformity of non-canonical substrate incorporation.

Because the Y639F mutation specifically reduces discrimination of the character of the ribose 2′-group, it is less useful for incorporating substrates modified at other positions (though non-specific effects due to a modest reduction in side-chain volume, structure perturbation, etc . . . cannot be ruled out). However, the active site of T7 RNAP has been heavily mutagenized (14, 16) and some of these mutations may, by reducing steric clashes with groups on modified substrates, be useful for incorporating particular substrates. A drawback to this approach is that these mutations tend to reduce activity and any gain in reduced substrate specificity may not compensate for the lower activity. We have found this to be the case in attempts to use Y639M or Y639L to incorporate substrates modified with bulky ribose 2′-groups. While only testing of particular substrates with particular mutants is likely to identify useful combinations, the outlined considerations on the mechanisms by which poly-amines, Mn⁺⁺, template structure and sequence, and substrate structure and concentration influence the transcription reaction should be useful in guiding development of methods for the enzymatic synthesis of modified nucleic acids.

All publications cited in the specification are incorporated by reference.

The present invention is not limited to the exemplified embodiments, but is intended to encompass all such modifications and variations as come within the scope of the following claims.

We claim:

1. A method of increasing the rate of transcript extension of a nucleic acid molecule comprising at least one non-canonical nucleotide comprising the steps of:
a) incubating a template nucleic acid in a reaction mixture under nucleic acid synthesis conditions, the reaction mixture containing (i) a mutant RNA polymerase, wherein the polymerase is selected from the group consisting of an SP6 RNA polymerase comprising an altered amino acid at position 631 and T7 RNA polymerase comprising an altered amino acid at position 639, wherein said polymerase has reduced discrimination between canonical and non-canonical nucleoside triphosphates, (ii) at least one non-canonical nucleoside triphosphate, wherein said non-canonical nucleoside triphosphate is incorporated into the synthesized nucleic acid in place of only one canonical nucleoside triphosphate, and (iii) a polyamine selected from the group consisting of spermidine at a concentration of at least about 8 mM and spermine at a concentration of at least about 1.5 mM; and
b) obtaining the synthesis of a nucleic acid molecule comprising at least one non-canonical nucleotide.

2. The method of claim 1, wherein the polyamine is spermidine present at a concentration of between about 8 mM and about 20 mM.

3. The method of claim 1, wherein the polyamine is spermidine present at a concentration of between about 8 mM and about 15 mM.

4. The method of claim 1, wherein the polyamine is spermine present at a concentration of between about 1.5 mM and about 15 mM.

5. The method of claim 1, wherein the rate of transcript extension is increased at least about 2-fold.

6. The method of claim 1, wherein the rate of transcript extension is increased at least about 5-fold.

7. The method of claim 1, wherein the rate of transcript extension is increased at least about 10-fold.

8. The method of claim 1, wherein the rate of transcript extension is increased at least about 20-fold.

9. The method of claim 1, wherein the rate of transcript extension is increased at least about 40-fold.

10. A buffer concentrate for performing the method of claim 1, comprising a polyamine selected from the group consisting of spermidine and spermine, wherein the spermidine or spermine is present at a concentration sufficient to give a final concentration of at least about 8 mM spermidine or at least about 1.5 mM spermine when the buffer is diluted to give suitable nucleic acid synthesis conditions.

11. A kit for performing the method of claim 1, comprising:
a mutant nucleic acid polymerase having reduced discrimination between canonical and non-canonical nucleoside triphosphates and a buffer comprising a polyamine selected from the group consisting of spermidine and spermine, present in an amount sufficient to give a concentration of at least about 8 mM spermidine or at least about 1.5 mM spermine when the buffer is diluted to give suitable nucleic acid synthesis conditions.

12. The kit of claim 11, wherein the polyamine is spermidine and wherein the protocol provides for a final concentration of spermidine of at least about 8 mM.

13. The kit of claim 11, wherein the polyamine is spermine and wherein the protocol provides for a final concentration of spermine of at least about 1.5 mM.
